(19) United States
(12) Patent Application Publication
(10) Pub. No.: US 2018/0280262 A1
(43) Pub. Date: Oct. 4, 2018

(54) COSMETIC PHOTOPROTECTION COMPOSITION

(71) Applicant: Rhodia Operations, Paris (FR)

(72) Inventors: Camille JOURDE, Paris (FR); Valérie BUISSETTE, Paris (FR)

(21) Appl. No.: 15/762,978

(22) PCT Filed: Sep. 23, 2016
(86) PCT No.: PCT/EP2016/072726

§ 371 Date: Mar. 23, 2018

(30) Foreign Application Priority Data
Sep. 25, 2015 (FR) 1559038

(51) Int. Cl. A61K 8/19; A61Q 17/04; A61K 8/02; A61K 8/81; A61K 8/27; A61K 8/29; A61K 8/06

(52) U.S. Cl. CPC A61K 8/19; A61Q 17/04; A61K 8/0241; A61K 8/8147; A61K 2800/614; A61K 8/29; A61K 8/06; A61K 2800/413; A61K 8/27

(57) ABSTRACT

The invention relates to a cosmetic photoprotection composition comprising particles of cerium oxide functionalised with polyacrylic acid, in combination with particles of titanium oxide or zinc oxide.

The present invention relates to a photoprotective cosmetic composition comprising cerium oxide particles functionalized with polyacrylic acid in combination with titanium oxide or zinc oxide particles.

### Technical Problem

Keratin materials, in particular the skin, are exposed to sunlight every day. It is known that prolonged exposure of keratin materials to sunlight is liable to induce skin disorders or surface damage. Radiation with wavelengths of between 280 nm and 400 nm permits tanning of the human epidermis and radiation with wavelengths between 280 and 320 nm, known as UV-B rays, harm the development of a natural tan.

UV-A rays with wavelengths of between 320 and 400 nm penetrate more deeply into the skin than UV-B rays. UV-A rays cause immediate and persistent browning of the skin. Daily exposure to UV-A rays, even for a short period, under normal conditions may lead to degradation of the collagen and elastin fibers, which is reflected by a change in the skin's microrelief, the appearance of wrinkles and non-uniform pigmentation (liver spots, or heterogeneity of the complexion).

Protection against UV-A and UV-B rays is thus necessary. An efficient photoprotective product must protect against both UV-A and UV-B rays. Photoprotective cosmetic compositions which screen out UV-A and UV-B rays already exist.

A photoprotective cosmetic composition which finds favor is one which, during and after spreading on the skin, has a pleasant feel. It is also preferable that the photocatalytic decomposition of the ingredients of the photoprotective cosmetic composition induced by the inorganic particles should be minimized.

The Applicant has developed a cerium oxide functionalized with polyacrylic acid (PAA) which may be used advantageously as inorganic UV-screening agent in a photoprotective cosmetic composition in combination with at least one other inorganic UV-screening agent based on TiO₂ or ZnO which meets the requirements described above.

### BRIEF DESCRIPTION OF THE INVENTION

According to a first subject, the invention relates to a photoprotective cosmetic composition in the form of an emulsion comprising:

a) the functionalized cerium oxide as described later;

b) at least one inorganic UV-screening agent based on titanium oxide or zinc oxide;

c) optionally at least one organic UV-screening agent.

More particularly, the photoprotective cosmetic composition may comprise, in a physiologically acceptable support:

i) at least one aqueous phase;

ii) at least one oily phase;

iii) at least one emulsifying system;

iv) the functionalized cerium oxide as described previously;

v) at least one inorganic UV-screening agent based on titanium oxide or zinc oxide;

vi) optionally at least one organic UV-screening agent.

According to a second subject, the invention relates to the use of functionalized cerium oxide as described later for the preparation of a photoprotective cosmetic composition in emulsion form.

According to yet another subject, the invention relates to the use of an aqueous-phase dispersion of particles of functionalized cerium oxide as described later for the preparation of a photoprotective cosmetic composition in emulsion form. The invention also relates to a photoprotective cosmetic composition obtained from such a dispersion.

### FIGURES

FIG. 1: electron microscopy photographs of functionalized cerium oxide particles of example 1.

FIG. 1bis: electron microscopy photograph of non-functionalized cerium oxide particles of example 1.

FIG. 2: diameter distribution of functionalized cerium oxide particles of example 1.

FIG. 3: curves of UV absorption (absorbance versus wavelength in nanometres) of the cosmetic composition of example 6 (5% by weight of functionalized cerium oxide of example 1+5% by weight of titanium oxide; curve 1), of example 3 (10% by weight of functionalized cerium oxide of example 1; curve 2) and of example 9 (10% by weight of titanium oxide; curve 3).

### PRIOR ART

US 2009/0162302 describes a composite powder comprising inorganic particles and a compound chosen from carboxylic acids and derivatives thereof, carboxylic acid polymers and derivatives thereof.

WO 00/14017 describes a process for preparing a modification of nanoparticles of a metal oxide, of a metal hydroxide and/or of a metal oxide-hydroxide, the metal being chosen from Al, Mg, Ce, Fe, Mn, Co, Ni, Cu, Ti, Zn and Zr, in which a polyacrylate and a solution of the metal are reacted in the presence of a base so as to obtain precipitation.

WO 03/072663 describes an organic cerium sol in which the particles have a size of not more than 200 nm, or even not more than 100 nm.

WO 03/099942 describes a composition based on a water-based paint based on cerium oxide particles, the size of which is not more than 200 nm.

WO 2008/043703 describes a suspension of cerium oxide particles in a liquid phase, the mean size of which is not more than 200 nm, these particles being constituted of primary particles whose mean size is not more than 100 nm. The suspension may be used as UV-screening agent in a cosmetic composition. Example 4 describes a suspension of cerium oxide modified with PAA with a median size d₅₀ of 97 nm.

WO 2010/020466 describes a suspension of cerium oxide particles in a liquid phase, characterized in that these particles (secondary particles) have a mean size of not more than 200 nm, these secondary particles being constituted of primary particles, the sizes of which measured by TEM have a mean value of not more than 150 nm with a standard deviation whose value is not more than 30% of the value of said mean size and for which the ratio of the mean size measured by TEM to the mean size measured by BET is at least 1.5. Example 2 describes a suspension of cerium oxide modified with PAA.

JP 2010/090056 describes a cosmetic composition which may contain mineral particles of the zinc oxide, titanium oxide or cerium oxide type. The cerium particles are different from those of claim 1.

US 2013/0189331 describes a powder whose surface is treated with a perfluoro product.

None of these documents describes or suggests a photoprotective cosmetic composition as claimed.

### Definitions

The term “photoprotective” is used in the present patent application to mean that the cosmetic composition, after application to a keratin material, can prevent or at least limit the contact of radiation with said surface via mechanisms of absorption and/or reflection and/or scattering of the UV-A and/or UV-B radiation.

The mean diameter d₅₀ corresponds to the median diameter of a distribution of particle diameters (weight distribution) obtained using a centrifugal sedimentation particle size analyzer. It is thus the value conventionally employed in statistics for which on the cumulative distribution curve, 50% of the particles have a diameter greater than d₅₀ and 50% of the particles have a diameter less than d₅₀. This particle size analyzer separates the particles as a function of their size by centrifugal sedimentation in liquid medium, the sedimentation being stabilized with a density gradient. The particles sediment by centrifugation in an optical-grade transparent disk and scatter a light beam. A BI-XDC machine from the company Brookhaven was used, following the manufacturer's recommendations, and a density of 7.2 was retained for cerium oxide.

The distribution dispersion index obtained by the centrifugal sedimentation particle size analyzer is defined by:

σ/m=(d₈₄−d₁₆)/2d₅₀

for which:
d₈₄ is the particle diameter for which 84% of the particles have a diameter smaller than d₈₄;
d₁₆ is the particle diameter for which 16% of the particles have a diameter smaller than d₁₆.

For the primary particles, the mean diameter (d_{TEM}) and the standard deviation (S_{TEM}) are calculated from a diameter distribution determined by means of transmission electron microscopy (TEM). The method consists in measuring the diameter of at least 150 and preferably at least 500 primary particles on one or more electron microscopy images. The microscope magnification that is selected must make it possible to clearly distinguish the particles on an image. The magnification may be, for example, between 50 000 and 500 000. The particle diameter that is selected is that of the minimum enclosing circle which can circumscribe the entirety of the image of the particle as is visible on a TEM image. The term “minimum enclosing circle” has the meaning given to it in mathematics and represents the circle of minimum diameter which can contain a set of points on a plane. Only the particles for which at least half of the perimeter is defined are selected. The ImageJ software may be used to perform the processing more simply: this open-access software was initially developed by the American NIH institute and is available at the following address: http://rsb.info.nih.gov or http://rsb.info.nih.gov/ij/download.html.

After having determined the diameters of the selected particles by the above method, said diameters are regrouped into several particle size categories ranging from 0 to 500 nm, the breadth of each category being 1 nm. The number of particles in each category is the basic data for representing the distribution by number (cumulative). The mean diameter d_{TEM} is the median diameter of the diameter distribution thus obtained (distribution by number). This median diameter is the one conventionally used in statistics such that 50% of the particles (by number) taken into account on the TEM image(s) have a diameter smaller than this value and 50% have a diameter larger than this value. From this distribution, it is also possible to determine the standard deviation S_{TEM}, which has the usual meaning used in mathematics and which may be defined as the square root of the variance:

$${S}_{TEM} = \sqrt{\frac{1}{n} {\sum }_{i = 1}^{n} {( {x}_{i} - \overline{x} )}^{2}}$$

n is the number of primary particles taken into account on the SEM image(s);

xᵢ is the diameter of a particle i on the SEM image(s);

x̅ is the mean diameter of the n primary particles, calculated according to the formula

1/nΣᵢ₌₁ⁿxᵢ.

The specific surface area is determined by nitrogen adsorption by application of the Brunauer-Emmett-Teller method which was described in J. Am. Chem. Soc. 1938, 60, page 309. The principle of this method is also described in ASTM D3663-03. The Flowsorb II 2300 machine from Shimadzu may be used to determine automatically the BET specific surface area according to the manufacturer's recommendations. From the BET specific surface area thus measured, it is possible to determine an equivalent diameter noted d_{BET} calculated from the following formula:

d_{BET} (in nm)=6000/(specific surface area in m²/g×mass per unit volume in g/cm³). For cerium oxide, a mass per unit volume of 7200 kg/cm³ is retained.

The term “emulsion” means any macroscopically homogeneous composition comprising at least two mutually immiscible phases; one being the dispersing continuous phase and the other being dispersed in said continuous phase in the form of droplets. The two phases are kinetically stabilized by at least one emulsifying system comprising at least one emulsifying surfactant.

The term “emulsifying surfactant” means any compound or mixture of compounds that is capable of increasing the kinetic stability of an emulsion. These compounds are generally amphiphilic and are surfactants characterized by their more or less hydrophilic or more or less lipophilic nature which will determine their ability to stabilize direct emulsions or reverse emulsions. They are especially classified by their HLB value according to the calculation method of W. C. Griffin in the document “Classification of Surface Active Agents by HLB, Journal of the Society of Cosmetic Chemists 1949, 311, 1” and in the document “Calculation of HLB of Non Ionic Surfactants, Journal of the Society of Cosmetic Chemists 1954, 249, 5”. Calculation of the HLB value according to this calculation method is performed according to the equation:

HLB=20×Mh/M

in which Mh is the molar mass of the hydrophilic part of the surfactant and M is the total molecular mass of the molecule.

### DETAILED DESCRIPTION OF THE INVENTION

As regards functionalized cerium oxide, it is composed of cerium oxide particles (secondary particles) with a mean diameter d₅₀ measured with a centrifugation particle size analyzer of between 35 and 300 nm, functionalized with polyacrylic acid (PAA), the weight-average molecular mass M_{w} of which is between 1500 and 4000 g/mol, preferably between 1800 and 2200 g/mol, said secondary particles being formed from aggregated primary particles with a mean diameter d_{TEM} of between 25 and 110 nm and a standard deviation S_{TEM} which is less than 30% of said mean diameter, d_{TEM} and S_{TEM} being calculated from a diameter distribution determined by means of transmission electron microscopy (TEM).

It is also possible to use a PAA comprising from 15 to 60 and preferably from 20 to 30 polymerized acrylic acid units.

The expression “functionalized with PAA” used in the present patent application to describe the particles means that the PAA is adsorbed onto the surface of these particles. This modification is obtained when the PAA interacts via chemical and/or physical bonds with the chemical groups at the surface of the particles.

Functionalization with PAA modifies the pH range in which the dispersion is stable. Specifically, the dispersion of non-functionalized cerium oxide, prepared via the process described below, is stable for a pH<6. The dispersion of functionalized cerium oxide is, for its part, stable for a pH >3, or better still >4. The dispersion according to the present invention preferably has a pH of between 5.0 and 9.5. The cosmetic compositions, for their part, generally have a pH of between 4 and 8, and as such the functionalized cerium oxide is suitable for use. It has also been found that the functionalized cerium oxide particles can be distributed both in the oily phase or at the edge of the oil droplets and in the aqueous phase of the emulsion.

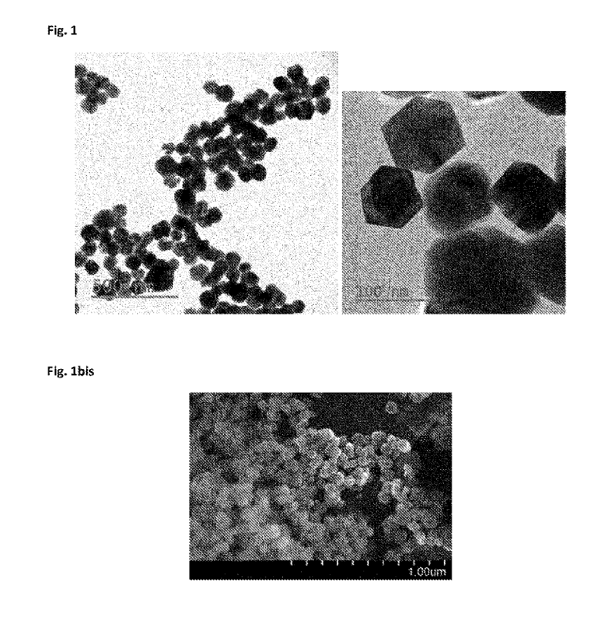

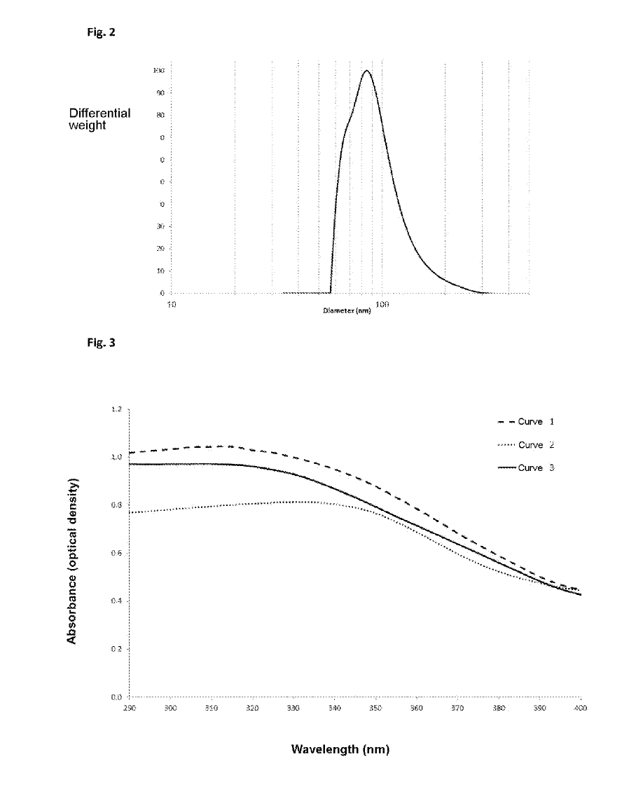

According to an important characteristic, the primary particles are fine and have a homogeneous diameter distribution. This may be demonstrated by means of the standard deviation S_{TEM} which is less than 30% of the mean diameter d_{TEM} (S_{TEM}<30%×d_{TEM}). It may preferably be less than 25% and more particularly less than 20% of said diameter d_{TEM}. This homogeneous nature is visible in the images of FIGS. 1 and 1bis.

The functionalized or non-functionalized cerium oxide particles may have, especially in the TEM images, a polygonal shape. This shape is regular. According to another embodiment, the diameter distribution d_{TEM} of the primary particles is a monopopulation.

The specific surface area according to the BET method for cerium oxide before functionalization is preferably between 7 and 45 m²/g. The functionalized and non-functionalized cerium oxide particles may have a d_{BET} of between 18.5 and 119.0 nm.

According to one embodiment, the secondary particles more particularly have a mean diameter d₅₀ of between 35 and 100 nm and the primary particles have a diameter d_{TEM} of about 30 nm or between 25 and 40 nm. For this embodiment, the functionalized and non-functionalized cerium oxide particles may have a d_{BET} of between 25 and 35 nm.

According to another embodiment, the secondary particles more particularly have a mean diameter d₅₀ of between 75 and 200 nm and the primary particles have a diameter d_{TEM} of about 60 nm or between 60 and 120 nm. For this embodiment, the functionalized and non-functionalized cerium oxide particles may have a d_{BET} of between 60 and 69 nm.

The mean diameter d₅₀ depends on the size of the primary particles of which the secondary particles are composed and also on the degree of aggregation thereof. The degree of aggregation itself depends, on the one hand, on the process used for the preparation of the non-functionalized cerium oxide particles and, on the other hand, on the process for modifying the surface of the particles.

According to one embodiment, the diameter distribution of the secondary particles is narrow and has a dispersion index (σ/m) of less than 0.50 and preferably less than 0.35. A narrow distribution is capable of leading to better dispersibility and a better feel of the cosmetic composition, once it has been applied to the skin.

The functionalized cerium oxide may be used in the form of a dry powder. Preferably, it is used in the form of a dispersion in an aqueous phase whose pH may be between 5.0 and 9.5. The aqueous phase contains water and optionally at least one water-soluble or water-miscible organic solvent. It is thus possible to use this dispersion directly during the preparation of the cosmetic composition. The use of a dry powder requires a redispersion step and has the risk of leading to aggregates.

Depending on the pH of the dispersion, the PAA is in acidic or basic form or else partly in acidic and basic form. The counteranion for the acid groups of the PAA may be, for example, K⁺, Na⁺ or NH₄⁺.

The proportion of functionalized cerium oxide in the dispersion may vary within a wide range, for example between 1% and 60% by weight, or even between 1% and 40% by weight, relative to the dispersion as a whole.

As regards the process for preparing the functionalized cerium oxide dispersion, it consists in first placing a dispersion in water of cerium oxide in contact with a solution of PAA in water. The pH of the dispersion comprising cerium oxide and PAA is generally between 3 and 4. Next, the pH of the dispersion comprising cerium oxide and PAA is raised using a base up to a pH of between 5.0 and 9.5. NaOH, KOH or NH₄OH may be used as base.

The contact time may range between 10 minutes and 2 hours; it may, for example, be between 20 and 40 minutes.

The dispersion of cerium oxide in water is obtained using a solution of Ce^{III} and Ce^{IV} according to the process described later. On conclusion of this process, the dispersion obtained has a pH<7 in a pH range extending from 1 to 6 (acidic dispersion). It is possible to use this acidic dispersion directly (see example 1 in which the dispersion is at a pH of 5.1). It is also possible, in a step prior to the placing in contact with PAA, to add a base to the acidic dispersion until the cerium oxide particles have precipitated, and then to separate the solid particles from the liquid medium. Precipitation takes place in the region of a pH value close to 7, or even greater than 7. The separation step may optionally be followed by a step of washing the particles with water. Redispersion of the particles after filtration and optional washing leads to a basic dispersion (pH >7).

The pH of this dispersion may be between 7 and 8. It is also possible to use this basic dispersion.

According to one embodiment, the cerium oxide dispersion (acidic or basic) is added with stirring to the PAA solution. According to another embodiment, the PAA solution is added with stirring to the cerium oxide dispersion (acidic or basic).

According to one embodiment, the dispersion obtained after placing the cerium oxide dispersion and the PAA in contact and raising the pH may be subjected to shear to deaggregate the particles. This step makes it possible to deaggregate the particles that have aggregated in one of the preceding steps. Use may be made, for example, in this step of a wet jet mill.

The added PAA does not become entirely attached to the surface of the particles. Specifically, only part of the PAA is adsorbed onto the surface of the cerium oxide particles and the remaining part stays in solution. The amount attached depends on the specific surface area and on the surface state of the cerium oxide. Tests have shown that for better stability of the dispersion, the amount of PAA (expressed in milligrams of PAA per g of cerium oxide) to be added is preferably at least 0.87×S_{BET} in which S_{BET} denotes the specific surface area in m²/g of the cerium oxide before modification. For example, an amount of between 0.87×S_{BET} and 1×S_{BET} may be added. The aqueous-phase dispersion that may be used for the preparation of the photoprotective cosmetic composition may thus comprise the functionalized cerium oxide particles as described previously and free PAA. Depending on the pH of the aqueous-phase dispersion, that which has been described previously for the adsorbed PAA remains valid for the free PAA, i.e. the free PAA is in acidic or basic form or else partly in acidic and basic form depending on the pH of the aqueous-phase dispersion.

As regards the process for preparing the cerium oxide dispersion used in the 1st step of the preceding process, it is described in international patent application WO 2008/043703. This process comprises the following steps:
(a) a solution of a cerium III salt which also comprises cerium IV is prepared;
(b) this solution is placed in contact under an inert atmosphere with a base, which produces a precipitate;
(c) the medium obtained in the preceding step is subjected to a heat treatment under an inert atmosphere, at least one of the steps (a), (b) or (c) being performed in the presence of nitrate ions;
(d) acidification and washing of the medium thus obtained are performed successively but in any order, which produces a dispersion.

The first step (a) of the above process thus consists in preparing a starting solution which is a solution of a Ce^{III} salt. As cerium III salts, use may be made more particularly of cerium III nitrate, chloride, sulfate or carbonate and also mixtures of these salts, such as mixed nitrate/chloride. In a known manner, this starting solution must have the appropriate acidity so that the cerium is fully present in solution. The starting solution also comprises Ce^{IV}. Ce^{IV} is provided by a salt which may be, for example, Ce^{IV} nitrate. The amount of Ce^{IV} is such that the (Ce^{IV}/Ce^{III}) mole ratio in the starting solution is between 1/90 000 and 1/50, or even between 1/80 000 and 1/1000, depending on the desired mean size.

The starting solution prepared in step (a) may be degassed beforehand by sparging with an inert gas. For the present description, the term “inert gas” or “inert atmosphere” means an atmosphere or a gas free of oxygen, the gas possibly being, for example, nitrogen or argon.

The second step (b) of the process consists in reacting the starting solution with a base. As base, use may be made especially of products of the hydroxide type. Mention may be made of alkali metal or alkaline-earth metal hydroxides and aqueous ammonia. Use may also be made of secondary, tertiary or quaternary amines. However, the amines and ammonia are preferred insofar as they reduce the risks of contamination with alkali metal or alkaline-earth metal cations. The base may also be degassed beforehand by sparging with an inert gas. To perform the reaction of the second step of the process, the placing in contact may take place in any order of introduction of the reagents. However, it is preferable to introduce the starting solution into a medium containing the base. This second step must be performed under an inert atmosphere, either in a closed reactor or in a semi-closed reactor while flushing with the inert gas. The placing in contact generally takes place in a stirred reactor. This second step is generally performed at room temperature (20-25° C.) or at a temperature of not more than 50° C.

The third step (c) of the process is a heat treatment of the reaction medium obtained on conclusion of the preceding step. This treatment consists in heating the medium and maintaining it at a temperature that is generally not more than 95° C. and more particularly between 60° C. and 95° C. The duration of this treatment may be between a few minutes and a few hours. This treatment is also performed under an inert atmosphere, that which was described on the subject of this atmosphere for the second step applying here similarly.

According to one characteristic of the process of the invention, at least one of the steps (a), (b) or (c) must be performed in the presence of nitrate ions. Generally, the nitrate ions are provided by the addition of nitric acid, more particularly in step (a), during the preparation of the cerium III solution. The amount of nitrate ions, expressed by the mole ratio NO₃⁻/Ce³⁺, is generally between 1/3 and 5.

The final step of the process, step (d), in fact comprises two successive operations which may be performed in any order. These operations are, firstly, acidification and, secondly, washing. These operations will be described below more precisely in the case of a sequence of acidification and then washing. The acidification generally takes place after cooling the medium obtained on conclusion of step (c) by addition of an acid. Any mineral or organic acid may be used. Nitric acid is used more particularly. The amount of acid added is such that the pH of the medium after acidification is between 1 and 5. This operation may be performed in air: it is no longer necessary to work under an inert atmosphere at this stage of the process.

The acidification is followed by washing, the purpose of which is to remove from the suspension the soluble species, essentially salts. The washing may be performed in various ways with or without solid/liquid separation. It may thus be performed by separating the solid particles from the liquid phase, for example by frontal filtration, decantation or centrifugation. The solid obtained is then resuspended in an aqueous phase. Tangential filtration may also be performed. This washing may optionally be repeated if necessary, for example until a given conductivity of the suspension is obtained, the conductivity measuring the amount of impurities present in this suspension. As indicated above, the order of the operations may be reversed relative to that which has just been described. Thus, on conclusion of step (c) and, in this case also, generally after cooling the medium obtained, washing may then be performed in the manner described above. On conclusion of the washing, the medium obtained is then acidified.

On conclusion of step (d), the cerium oxide dispersion which must be functionalized is obtained.

A second embodiment of the process will now be described. This second mode differs from the first solely by the first step. This first step consists in preparing a solution of a Ce^{III} salt which also comprises aqueous hydrogen peroxide solution. The description given above regarding the nature of the Ce^{III} salt applies equally here. The amount of H₂O₂ solution is such that the (H₂O₂/Ce^{III}) mole ratio in the cerium salt solution is between 1/10 000 and 1/100. The remainder of the process according to this second mode proceeds as described above for the first mode, i.e. the solution from the first step is placed in contact with a base under an inert atmosphere, a heat treatment is performed under an inert atmosphere and the medium thus obtained is acidified and washed (steps (b), (c) and (d) as described above with presence of nitrate ions in at least one of the steps (a), (b) and (c)). The description given above for all of these subsequent steps and for the first embodiment of the process thus applies equally here for the second mode.

The dispersion of unmodified cerium oxide may be a Zenus brand dispersion sold by the company Solvay.

As regards the photoprotective cosmetic composition, it is obtained from the aqueous-phase dispersion of the functionalized cerium oxide particles. It is in the form of an emulsion and it comprises:

a) the functionalized cerium oxide as described previously;

b) at least one inorganic UV-screening agent based on titanium oxide or zinc oxide;

c) optionally at least one organic UV-screening agent.

The photoprotective cosmetic composition may comprise, in a physiologically acceptable support:

i) at least one aqueous phase;

ii) at least one oily phase;

iii) at least one emulsifying system;

iv) the functionalized cerium oxide as described previously;

v) at least one inorganic UV-screening agent based on titanium oxide or zinc oxide;

vi) optionally at least one organic UV-screening agent.

The emulsion may be of the oil-in-water type (i.e. a cosmetically acceptable support constituted of an aqueous dispersing continuous phase and an oily dispersed discontinuous phase) or of the water-in-oil type (i.e. a cosmetically acceptable support constituted of an oily dispersing continuous phase and an aqueous dispersed discontinuous phase). It may also be a multiple emulsion, for example of the water-in-oil-in-water or oil-in-water-in-oil type.

The aqueous phase of the cosmetic composition contains water and optionally other water-soluble or water-miscible organic solvents. The water-soluble or water-miscible solvents comprise monoalcohols with a short chain, for example of C₁-C₄, for instance ethanol or isopropanol; diols or polyols, for instance ethylene glycol, 1,2-propylene glycol, 1,3-butylene glycol, hexylene glycol, diethylene glycol, dipropylene glycol, 2-ethoxyethanol, diethylene glycol monomethyl ether, triethylene glycol monomethyl ether, glycerol and sorbitol, and mixtures thereof. According to a preferred embodiment, ethanol, propylene glycol, glycerin, and mixtures thereof, may be used more particularly.

The oily phase of the cosmetic composition comprises one or more fatty substances, these fatty substances possibly being constituted by an oil or a wax or mixtures thereof. The term “oil” means a compound that is liquid at room temperature. The term “wax” means a compound that is solid or substantially solid at room temperature, and whose melting point is generally greater than 35° C. The oil may be advantageously chosen from the group constituted by mineral oils; natural oils, for instance castor oil; plant oils, for instance sweet almond oil, macadamia oil, blackcurrant pip oil or jojoba oil; synthetic oils, for instance perhydrosqualene and fatty alcohols, acids or esters; silicone oils. Among the fatty esters, mention may be made of C₁₂-C₁₅ alkyl benzoates, octyl palmitate, isopropyl lanolate, and triglycerides including those of capric or caprylic acids.

The emulsifying system comprises at least one surfactant which may be anionic, cationic, nonionic or amphoteric or a mixture of these compounds and optionally at least one cosurfactant. The emulsifying system is chosen according to the nature of the emulsion. The proportion of surfactant and of cosurfactant generally ranges from 0.3% to 20% by weight of the cosmetic composition.

In the cosmetic composition, the PAA is in acidic or basic form or else partly in acidic and basic form. The counteranion for the acid groups of the PAA may be, for example, K⁺, Na⁺ or NH₄⁺.

The nature and amount of each UV-screening agent used in the cosmetic composition are selected as a function of the desired sun protection factor, and also of the nature of the emulsion and of its ingredients. The weight proportion of functionalized cerium oxide may range from 0.5% to 40%, preferably from 1% to 30% or even from 1% to 10% of the total weight of the cosmetic composition (proportion X). The weight proportion of the inorganic UV-screening agent based on titanium oxide or zinc oxide may range from 0.5% to 40%, preferably from 1% to 30% of the total weight, or even from 1% to 10%, of the cosmetic composition (proportion Y). The total proportion of the inorganic UV-screening agents is preferably limited to 40%, or even 30%, of the total weight of the cosmetic composition. The organic UV-screening agent optionally present in the cosmetic composition represents from 0.1% to 30% and preferably from 1% to 25% of the total weight of the cosmetic composition.

The organic UV-screening agent may be chosen from water-soluble organic screening agents, and organic screening agents that are liposoluble or insoluble in the solvents commonly used in photoprotective compositions. Among the organic UV-screening agents that may be distinguished are UV-B screening agents which absorb UV radiation over a wavelength range of between 280 and 320 nm and UV-A screening agents which absorb between 320 and 400 nm. The organic screening agents are for the most part liposoluble. The UV-B screening agents most commonly used are cinnamates, benzotriazoles, salicylates, octocrylene, phenylbenzimidazolesulfonic acid, ethylhexyl triazone, diethylhexyl butamido triazone, camphor derivatives and benzophenones. The UV-A screening agents commonly used are dibenzoylmethane, diethylaminohydroxybenzoylhexyl benzoate and terephthalylidene dicamphor acid. It is also possible to use broad-spectrum organic screening agents: bis(ethylhexyloxyphenol)methoxyphenyltriazine (Tinosorb S®) and methylenebis(benzotriazolyl)tetramethylbutylphenol (Tinosorb M®) which have the particular feature of covering a broad spectrum of absorption in UV-B and UV-A.

It may be chosen especially from cinnamic derivatives; anthranilates; salicylic derivatives; dibenzoylmethane derivatives, especially avobenzone; camphor derivatives; benzophenone derivatives; β,β-diphenylacrylate derivatives; triazine derivatives; benzotriazole derivatives; benzalmalonate derivatives, especially those mentioned in patent U.S. Pat. No. 5,624,663; benzimidazole derivatives; imidazolines; bis-benzazolyl derivatives as described in patents EP 669 323 and U.S. Pat. No. 2,463,264; p-aminobenzoic acid (PABA) derivatives; methylenebis-(hydroxyphenylbenzotriazole) derivatives as described in U.S. Pat. No. 5,237,071, U.S. Pat. No. 5,166,355, GB 2303549, DE 19726184 and EP 893119; benzoxazole derivatives as described in EP 0832642, EP 1027883, EP 1300137 and DE 10162844; screening polymers and screening silicones such as those described especially in patent application WO 93/04665; α-alkylstyrene-based dimers, such as those described in DE 198 55 649; 4,4-diarylbutadienes such as those described in EP 0967200, DE 19746654, DE 19755649, EP-A-1008586, EP 1133980 and EP133981, merocyanin derivatives such as those described in WO 04/006878, WO 05/058269 and WO 06/032741; and mixtures thereof; the indanylenes described in EP-A-0823418 and EP-A-1341752, and mixtures thereof. It may also be chosen from the organic UV-screening agents described in the examples.

As examples of organic UV-screening agents that may be used, mention may be made of the following compounds: 1-(4-methoxyphenyl)-3-(4-tert-butylphenyl)propane-1,3-dione (or avobenzone); [(3Z)-3-[[4-[(Z)-[7,7-dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid; 2-(2H-benzotriazol-2-yl)-4-methyl-6-[2-methyl-3-[1,3,3,3-tetramethyl-1-[(trimethylsilyl)oxy]-1-disiloxanyl]propyl]phenol; 2-ethylhexyl 2-cyano-3,3-diphenyl-2-propenoate (or octocrylene); 3,3,5-trimethylcyclohexyl 2-hydroxybenzoate (or homosalate); 2-phenyl-3H-benzimidazole-5-sulfonic acid (or insulizole); (RS)-2-ethylhexyl (2E)-3-(4-methoxyphenyl)prop-2-enoate (or octinoxate); 2-ethylhexyl 4-(dimethylamino)benzoate (or octyldimethyl PABA); 2,2′-[6-(4-methoxyphenyl)-1,3,5-triazine-2,4-diyl]bis{5-[(2-ethylhexyl)oxy]phenol} (or bemotrizinol); bis(ethylhexyloxyphenol)methoxyphenyltriazine; ethylhexyl triazone; terephthalylidenedicamphorsulfonic acid (or Mexoryl SX), drometrizole trisiloxane (or Mexoryl XL); 3-(4′-methylbenzylidene)-dl-camphor (or Eusolex 6300); 3-benzylidenecamphor (or Mexoryl SD); N,N,N-trimethyl-4-(2-oxoborn-3-ylidenemethyl)anilinium methyl sulfate; ethoxylated ethyl 4-aminobenzoate. Use may also be made of Tinosorb M® (methylenebis(benzotriazolyl)tetramethylbutylphenol), which is an organic screening agent in solid form, which is neither liposoluble nor water-soluble, and which disperses in the cosmetic composition. This organic UV-screening agent is characterized by absorption similar to that of the other organic screening agents, but also by its property of reflecting and scattering light like an inorganic screening agent.

For its part, the inorganic UV-screening agent based on TiO₂ or ZnO is in the form of zinc oxide or titanium oxide particles. The median diameter d₅₀ of these particles may be less than 400 nm. The primary particle size of TiO₂ determined by x-ray is preferably less than 40 nm. The primary particle size of ZnO may, for its part, be less than 100 nm.

The particles may have undergone a surface treatment of chemical, electronic, mechanochemical and/or mechanical nature with compounds such as amino acids, beeswax, fatty acids, fatty alcohols, anionic surfactants, lecithins, sodium, potassium, zinc, iron or aluminum salts of fatty acids, metal alkoxides (of titanium or aluminum), polyethylene, silicones, proteins (collagen, elastin), alkanolamines, silicon oxides, metal oxides or sodium hexametaphosphate.

The titanium oxide may be covered with silica. The proportion of silica may range from 8% to 30% by weight, more particularly from 12% to 20% by weight, of the total amount of titanium oxide+silica. The silica layer covering the titanium oxide may be obtained via a sol-gel process by placing a silicate solution and a titanium oxide particle dispersion in contact at a temperature close to 80° C. and at a pH of about 6-7. A process for preparing silica-covered titanium oxide is described in patent application US 2006/0194057, especially in examples 2a, 2b and 2c.

The titanium oxide may be optionally doped with at least one transition metal, for instance iron or manganese. Reference may be made, for example, to patent application WO 2015/0876637.

The titanium oxide may be in amorphous or crystalline form. It may be predominantly in rutile and/or anatase form. The rutile form is preferred for better photostability of the photoprotective cosmetic composition.

As examples of titanium oxide, mention may be made of the following products, which are commercial: MT-100TV, Microtitanium Dioxide MT 500 B or Microtitanium Dioxide MT600 B from the company Tayca; “PW transparent oxide” from the company Wacker; Tioveil AQ from the company Tioxide. They may also be the following titanium oxides sold by the company Merck: Eusolex T-2000, Eusolex T-AQUA, Eusolex T-AVO as described in the examples or Eusolex T-OLEO.

According to a particular embodiment, the cosmetic composition comprises a silica-covered titanium oxide as inorganic UV-screening agent other than the functionalized cerium oxide.

The photoprotective cosmetic composition may comprise other additives that are common in the cosmetic field. These may be, for example, vitamins or precursors or derivatives thereof, dyestuffs, thickeners, emollients, antioxidants, fragrances, gelling agents or matt-effect agents.

Combination of Inorganic UV-Screening Agents

The photoprotective cosmetic composition thus combines two inorganic UV-screening agents. The Applicant has found that it is possible to combine the two inorganic UV-screening agents so as to reduce the total amount of inorganic screening agents without degrading the UV absorption. Thus, the cosmetic composition according to the invention may have a UV absorption in the 290-400 nm range greater than that of a photoprotective cosmetic composition comprising, in the same weight proportions, exactly the same ingredients other than the inorganic UV-screening agents, but comprising as inorganic UV-screening agent only the inorganic UV-screening agent based on titanium oxide or zinc oxide in a percentage weight proportion (X+Y).

In other words, for the two compositions 1 and 2 below:
composition 1: photoprotective cosmetic composition comprising X % by weight of functionalized cerium oxide and Y % by weight of the inorganic UV-screening agent other than the functionalized cerium oxide;
composition 2: photoprotective cosmetic composition comprising (X+Y)% by weight of the inorganic UV-screening agent other than the functionalized cerium oxide;
   this gives: absorbance_{290-400 nm} for composition 1>absorbance_{290-400 nm} for composition 2.

In the case where the cosmetic composition comprises more than one inorganic UV-screening agent other than the functionalized cerium oxide, it is possible to obtain the same effect. In this case, the percentage weight proportion of each inorganic UV-screening agent, noted UVi, other than the functionalized cerium oxide, may be noted as Yi, i being an integer greater than 2 (i>2). The weight proportion of the inorganic UV-screening agents other than the functionalized cerium oxide is thus Y % by weight=Yi representing the total proportion of all the inorganic UV-screening agents other than the functionalized cerium oxide:
composition 1: photoprotective cosmetic composition comprising X % by weight of functionalized cerium oxide and Yi % by weight of each inorganic UV-screening agent UVi other than the functionalized cerium oxide;
composition 2: photoprotective cosmetic composition comprising (X Yi/Σ Yi+Yi)% by weight of each inorganic UV-screening agent UVi other than the functionalized cerium oxide;
   this gives: absorbance_{290-400 nm} for composition 1>absorbance_{290-400 nm} for composition 2. The absorbance values are measured according to standard ISO 24443 (edition 2012-06-01).

The emulsion may be stable when the composition is subjected to each of the following tests:

4 weeks, 4° C.
4 weeks, room temperature
4 weeks, 40° C.
10 days, 50° C.
1 week, cycles: 12 hours at +20° C., then 12 hours at −10° C.
1 week, cycles: 12 hours at +40° C., then 12 hours at −10° C.

The term “stable emulsion” means that the emulsion does not become destructured.

The absorbance is obtained on a thin film of the cosmetic composition deposited on a PMMA plate, one face of which is of controlled roughness which must be transparent and non-fluorescent. The products are applied to the rough face. The roughness of the plate is as defined in standard ISO 24443 (edition 2012-06-01): arithmetic roughness Rₐ of between 4.535 and 5.170 μm; minimum valley depth Rᵥ of between 12.414 and 13.669 μm; mean profile gradient R_{dq} of between 9.833 and 12.411°, A₁ between 195.244 and 284.256 μm²/mm; SS_{c} between 0.020 and 0.046; V_{W} between 4.248×10⁻⁷ and 1.663×10⁻⁶ mL/mm². These parameters are defined in Table I of the article “Sandblasting to Improve the Reproducibility of In vitro Sunscreen Evaluation” Cosmetics & Toiletries, Science Applied, S. Miksa, D. Lutz, C. Guy, 20 Mar. 2014.

The cosmetic composition may be prepared by mixing the aqueous phase comprising the functionalized cerium oxide and the oily phase comprising the inorganic UV-screening agent other than the functionalized cerium oxide and subjecting the mixture thus obtained to shear. The term “shear” means that the mechanical energy released by the stirring rotor and which is applied to the mixture allows the creation of kinetically stable droplets. These droplets are those of the aqueous phase or of the oily phase depending on the nature of the emulsion.

According to one embodiment, the aqueous phase is added to the oily phase under shear and portionwise.

In the presence of ingredients that are solid at room temperature, it may be necessary to liquefy the oily phase by heating. The shear may be obtained using a machine of Ultra-Turrax type. A person skilled in the art may use the process described in examples 6 to 9.

According to another subject, the invention relates to the use of functionalized cerium oxide as described previously for the preparation of a photoprotective cosmetic composition in emulsion form, in particular in the form of a stable emulsion. According to yet another subject, the invention relates to the use of an aqueous dispersion of the functionalized cerium oxide as described previously, for the preparation of a photoprotective cosmetic composition in emulsion form, in particular in the form of a stable emulsion.

### EXAMPLES

### Example 1—Preparation of a Dispersion of Functionalized Cerium Oxide

A cerium oxide dispersion sold under the brand name Zenus® HC60 (non-functionalized cerium oxide) was used. The characteristics of the batch used are as follows:
it is an acidic dispersion whose pH is about 5.1;
weight proportion of cerium oxide: 30%;
d₅₀ (BI-XDC centrifugal sedimentation particle size analyzer from Brookhaven; density of 7.2 for cerium oxide): 87.0 nm;
σ/m (BI-XDC)=0.33;
d₁₆=70 nm; d₈₄=128 nm;
mean primary particle diameter determined by TEM: d_{TEM}=89.0 nm;
standard deviation S_{TEM}: 18.0 nm, i.e. 20%;
BET surface area: 13.6 m²/g.

d_{TEM} and S_{TEM} were determined by the method given in the “Definitions” section. A JEOL 1440 TEM microscope equipped with an Orius 1000 2 k-2 k camera (4 megapixels) was used. According to this method, one drop of the particle dispersion subjected beforehand to an ultrasonic treatment (15 minutes under 30 W of ultrasound) is deposited on two carbonized gratings that have been rendered hydrophilic beforehand and the liquid of the dispersion is allowed to evaporate off. The carbonized grating is a carbon formvar membrane on a 200 mesh copper grating from Pelco. The hydrophilization treatment renders the support hydrophilic and negatively charged. It may be performed using an Elmo air glow discharge system from Cordouan Technologies.

The pH of the Zenus® HC60 cerium oxide dispersion (1 liter) is increased by adding 1M sodium hydroxide until a pH value of 9 is obtained to make the cerium oxide precipitate. The solid is then washed by adding deionized water and 500 mL of a dispersion containing 35.9% by weight of cerium oxide whose pH is 7.5 is recovered (there are 256 g of solid).

The amount of PAA in milligrams to be added is 256×13.6×0.87=3.03 g. 6.06 g of an aqueous PAA solution sold by Aldrich (M_{w}=2000 g/mol; 50% by weight of PAA, i.e. 3.03 g of PAA) is added to this dispersion. The pH is then increased to a pH value of 8.5 using a 1M sodium hydroxide solution. A functionalized cerium oxide dispersion of pH=8.5 is thus obtained, the weight proportion of which is adjusted to 30%.

Characteristics of the Functionalized Particles
d₅₀ (BI-XDC, density of 7.2): 154.0 nm;
σ/m (BI-XDC)=0.40;
mean primary particle diameter determined by TEM: d_{TEM}=89.0 nm;
standard deviation S_{TEM}: 18.0 nm, i.e. 20%;

As is visible in the SEM photos of FIG. 1, it may be seen that the primary particles have great size and shape homogeneity. Their polygonal shape may also be noted.

### Examples 2-5: Stability Over Time of Photoprotective Cosmetic Compositions

These examples correspond to stability tests. The photoprotective cosmetic compositions were prepared on a scale of about 300 g using the ingredients of Table I (the proportions are given as weight percentages of the total composition) using tools that are common in cosmetics formulation.

TABLE I

| | | | | | |
|---|---|---|---|---|---|
| | | Ex. 2 | Ex. 3 | | |
| | | 5% | 10% | Ex. 4 | Ex. 5 |
| | INCI (EU)/ | CeO₂- | CeO₂- | 5% | 10% |
| Ingredient | supplier | PAA | PAA | CeO₂ | CeO₂ |

Phase A

| | | | | | | |
|---|---|---|---|---|---|---|
| 1 | Amphisol K | Potassium cetyl | 2 | 2 | 2 | 2 |
| | | phosphate/ | | | | |
| | | Nordmass | | | | |
| | | Rassmann | | | | |
| 2 | Lanette O | Cetearyl alcohol/ | 2 | 2 | 2 | 2 |
| | | BTC Europe | | | | |
| | | GmbH | | | | |
| 3 | Tegosoft TN | C12-C15 alkyl | 10 | 10 | 10 | 10 |
| | | benzoate/Evonik | | | | |
| | | Industries AG | | | | |
| 4 | Isopropyl | Isopropyl | 5 | 5 | 5 | 5 |
| | palmitate | palmitate/ | | | | |
| | | BTC Europe | | | | |
| | | GmbH | | | | |
| 5 | Myritol 331 | Cocoglycerides/ | 5 | 5 | 5 | 5 |
| | | BTC Europe | | | | |
| | | GmbH | | | | |
| 6 | Euxyl PE 9010 | Phenoxyethanol, | 1 | 1 | 1 | 1 |
| | | ethylhexyl- | | | | |
| | | glycerin, | | | | |
| | | tocopherol/ | | | | |
| | | Schulke & Mayr | | | | |

Phase B

| | | | | | | |
|---|---|---|---|---|---|---|
| 7 | Water | Aqua | 53.85 | 37.25 | 52.2 | 33.7 |
| 8 | Glycerin | Glycerin, aqua/ | 4 | 4 | 4 | 4 |
| | Ph Eur 85% | Azelis | | | | |
| 9 | Keltrol | Xanthan gum/ | 0.3 | 0.3 | 0.3 | 0.3 |
| | CG-SFT | Rahn AG | | | | |
| 10 | Functionalized | Aqua, cerium | 16.7 | 33.3 | — | — |
| | cerium oxide of | oxide/Solvay | | | | |
| | example 1 | | | | | |
| 11 | Non- | Aqua, cerium | — | — | 18.5 | 37.0 |
| | functionalized | oxide/Solvay | | | | |
| | cerium oxide | | | | | |
| | (Zenus ® | | | | | |
| | HC60) | | | | | |
| 12 | Citric acid | Citric acid, aqua/ | 0.15 | 0.15 | — | — |
| | (50% in water) | Merck | | | | |

Phase C

| | | | | | | |
|---|---|---|---|---|---|---|
| 13 | Citric acid | Citric acid, aqua/ | — | — | qs | — |
| | (50% in water) | Merck | | | | |
| 14 | NaOH (10% | Aqua, sodium | — | — | qs | qs |
| | aq.) | hydroxide | | | | |

qs = quantum satis;
CeO₂-PAA: functionalized cerium oxide of example 1

for ingredients 10 and 11, the percentage indicated corresponds to the weight of the solid

Preparation of the Phases Noted A-B
phase A: the ingredients of phase A are heated to about 85° C. to liquefy them;
phase B: ingredients 8 and 9 are dispersed homogeneously in ingredient 7 for 20 minutes at room temperature. The dispersion of the invention (ingredient 10) or the dispersion Zenus® HC60 (ingredient 11), is then added to the mixture of ingredients 7-8-9 with stirring. In the case of preparations with the suspension of the invention, citric acid 12 is added with stirring. The mixture is heated to about 80° C.

Preparation of the Cosmetic Compositions

The liquid phase B is added with vigorous stirring (2000 rpm with a mechanical stirrer) portionwise to phase A which is heated. Once the mixture has been prepared, stirring is continued for 1 minute.

The emulsion obtained is then cooled to about 25° C. and left under gentler stirring (paddle stirrer; 130 rpm) and then homogenized by treatment with a Homozenta emulsifier (manufacturer: Swiss company Zehnder). The pH is then adjusted to the desired value using citric acid and/or sodium hydroxide (phase C). The air is then stripped out by applying a vacuum.

Heat stability results: ✓=the formulation is stable at the end of the test, x=the formulation shows a change in physicochemical properties and/or in characterization of the emulsion (instability, viscosity, structure of the emulsion, color, etc.)

TABLE II

| | | | Non- | |
|---|---|---|---|---|
| | Cerium oxide of | | functionalized | |
| | example 1 | | cerium oxide | |
| | Ex. 2 | Ex.3 | Ex. 4 | Ex. 5 |
| | 5% by | 10% by | 5% by | 10% by |
| | weight | weight | weight | weight |
| 4 weeks, 4° C. | ✓ | ✓ | x | x |
| 4 weeks, room temperature | ✓ | ✓ | ✓ | x |
| 4 weeks, 40° C. | x | x | x | x |
| 10 days, 50° C. | ✓ | ✓ | ✓ | x |
| 1 week, cycles: 12 hours | ✓ | ✓ | x | ✓ |
| at +20° C., then 12 hours | | | | |
| at −10° C. | | | | |
| 1 week, cycles: 12 hours | ✓ | ✓ | x | x |
| at +40° C., then 12 hours | | | | |
| at −10° C. | | | | |

The compositions comprising cerium oxide functionalized with PAA show better stability than those comprising non-functionalized cerium oxide. This observation may also be made for the compositions comprising the two inorganic UV-screening agents according to the invention.

### Examples 6-9: Combination of Functionalized Cerium Oxide and Titanium Oxide

These examples correspond to stability tests and to UV absorption measurements. The photoprotective cosmetic compositions were prepared on a scale of about 300 g using the ingredients of Table III (the proportions are given as weight percentages of the total composition) using tools that are common in cosmetics formulation.

TABLE III

| | | | | | |
|---|---|---|---|---|---|
| | | Ex. 6 | Ex. 7 | | |
| | | (inv.) | (comp.) | | |
| | | 5% CeO₂- | 5% | Ex. 8 | Ex. 9 |
| | | PAA + | CeO₂ + | (comp.) | (comp. |
| | | 5% | 5% | 5% | 10% |
| Ingredient | INCI (EU)/supplier | TiO₂ | TiO₂ | TiO₂ | TiO₂ |

Phase A

| | | | | | | |
|---|---|---|---|---|---|---|
| 1 | Amphisol K | Potassium cetyl | 2 | 2 | 2 | 2 |
| | | phosphate/Nordmass | | | | |
| | | Rassmann | | | | |
| 2 | Lanette O | Cetearyl alcohol/BTC | 2 | 2 | 2 | 2 |
| | | Europe GmbH | | | | |
| 3 | Tegosoft TN | C12/C15 alkyl | 10 | 10 | 10 | 10 |
| | | benzoate/Evonik Ind. | | | | |
| 4 | Isopropyl | Isopropyl palmitate/ | 5 | 5 | 5 | 5 |
| | palmitate | BTC Europe GmbH | | | | |
| 5 | Myritol 331 | Cocoglycerides/BTC | 5 | 5 | 5 | 5 |
| | | Europe GmbH | | | | |
| 6 | Euxyl PE | Phenoxyethanol, | 1 | 1 | 1 | 1 |
| | 9010 | ethylhexylglycerin, | | | | |
| | | tocopherol/Schulke & | | | | |
| | | Mayr | | | | |

Phase B

| | | | | | | |
|---|---|---|---|---|---|---|
| 7 | Eusolex T | TiO₂ (nano) covered | 5 | 5 | 5 | 10 |
| | AVO | with SiO₂; | | | | |
| | | predominantly rutile, | | | | |
| | | particles d₅₀ <200 nm/ | | | | |
| | | Merck | | | | |

Phase C

| | | | | | | |
|---|---|---|---|---|---|---|
| 8 | Water | Aqua | 48.85 | 47.20 | 65.55 | 60.55 |
| 9 | Glycerin Ph | Glycerin, aqua/Azelis | 4 | 4 | 4 | 4 |
| | Eur 85% | | | | | |
| 10 | Keltrol CG- | Xanthan gum/Rahn | 0.3 | 0.3 | 0.3 | 0.3 |
| | SFT | AG | | | | |
| 11 | Dispersion | Aqua, cerium oxide/ | 16.7 | — | — | — |
| | example 1 | Solvay | | | | |
| 12 | Non- | Aqua, cerium oxide/ | — | 18.5 | — | — |
| | functionalized | Solvay | | | | |
| | CeO₂ | | | | | |
| | (Zenus ® | | | | | |
| | HC60) | | | | | |
| 13 | Citric acid | Citric acid, aqua/ | 0.15 | — | 0.15 | 0.15 |
| | (50% in | Merck | | | | |
| | water) | | | | | |

Phase D

| | | | | | | |
|---|---|---|---|---|---|---|
| 14 | Citric acid | Citric acid, aqua/ | — | — | qs | — |
| | (50% in | Merck | | | | |
| | water) | | | | | |
| 15 | NaOH (10% | Aqua, sodium | — | qs | — | — |
| | aq.) | hydroxide | | | | |

inv. according to the invention;
comp. comparative;
CeO₂-PAA: functionalized cerium oxide of example 1

Preparation of the Phases Noted A-C
phase A: the ingredients of phase A are heated to about 85° C. to liquefy them;
phase B: addition to phase A of the inorganic UV-screening agent other than the functionalized cerium oxide, ingredient 7;
phase C: ingredients 9 and 10 are dispersed homogeneously in ingredient 8 for 20 minutes at room temperature. When it is used, the dispersion of the invention (ingredient 11) or the dispersion Zenus® HC60 (ingredient 12) is then added to the mixture of ingredients 8-9-10 with stirring. In the case of preparations with the suspension of the invention, citric acid 13 is added with stirring. The mixture is heated to about 80° C.

Preparation of the Cosmetic Compositions

The liquid phase C is added with vigorous stirring (2000 rpm) portionwise to phases A+B which are heated. Once the mixture has been prepared, stirring is continued for 1 minute.

The emulsion is then cooled to about 25° C. and left under gentler stirring (paddle stirrer; 130 rpm) and then homogenized by treatment with a Homozenta emulsifier (manufacturer: Swiss company Zehnder). The pH is then adjusted to the desired value using citric acid and/or sodium hydroxide (phase D). The air is then stripped out by applying a vacuum.

Heat stability results: ✓=formulation stable at the end of the test, x=change in physicochemical properties and/or in characterization of the emulsion (instability, viscosity, structure of the emulsion, color, etc.)

TABLE IV

| | Ex. 6 | | |
|---|---|---|---|
| | 5% CeO₂- | Ex. 7 | |
| | PAA + | 5% CeO₂ + | Ex. 8 |
| | 5% TiO₂ | 5% TiO₂ | 5% TiO₂ |
| 4 weeks, 4° C. | ✓ | ✓ | ✓ |
| 4 weeks, room temperature | ✓ | ✓ | ✓ |
| 4 weeks, 40° C. | ✓ | ✓ | ✓ |
| 10 days, 50° C. | ✓ | ✓ | ✓ |
| 1 week, cycles: 12 hours | ✓ | x | x |
| at +20° C., then 12 hours | | | |
| at −10° C. | | | |
| 1 week, cycles: 12 hours | ✓ | ✓ | ✓ |
| at +40° C., then 12 hours | | | |
| at −10° C. | | | |

It is observed that the combination according to the invention makes it possible to obtain a stable cosmetic composition, even for a total proportion of inorganic UV-screening agents of 10% by weight. The functionalized cerium oxide according to the invention may be used to stabilize the photoprotective cosmetic composition as defined previously, especially in claims 1 to 3.

UV Absorbance Measurement

This involves measuring the UV transmittance through a thin film of the composition spread on a UV-transparent rough support. The measurement is performed using a Bentham SSUV300 spectrometer (transmission measurement) on a thin film of the sample to be tested deposited onto a PMMA plaque of controlled roughness. The detection comprises an integration head and a double monochromator equipped with a photomultiplier, for minimizing the influence of the scattered light. The measurement is taken over the range 290-400 nm with an increment of 1 nm. The measurement reference is the same type of rough PMMA plaque onto which is deposited a thin film of glycerin not containing any scattering particles. After application, the plaques are left at equilibrium in the dark for about 20 minutes at room temperature before measuring the adsorption.

Production of the Thin Film on the PMMA Plaque
application of the emulsion on a PMMA plaque (PMMA 120 sold by the company Schönberg GmbH & Co KG, Hamburg, Germany; 50×50×2.5 mm; area: 25 cm²; roughness Ra=4.853; 1 single rough face at the top);
amount applied: 1.3 mg/cm²±2% using a micropipette, then spread by finger. The amount of product applied to a plaque is about 32.5 mg;
number of measurements for each composition evaluated: 12 (4 measurements taken for each plaque; there are 3 plaques for each composition). The curves of FIG. 3 thus correspond to an arithmetic mean.

Curves 1-3 of FIG. 3 correspond to the cosmetic compositions 6, 3 and 9. It may be observed that the absorbance of composition 6 shows better absorption throughout the range 290-400 nm than composition 3 or 9. In particular, it is possible to observe that for the same proportion of inorganic particles (10%), the combination of the two inorganic UV-screening agents (functionalized cerium oxide+titanium oxide) shows better absorption of the titanium oxide.

Application of the photoprotective cosmetic composition of example 3 to the skin: the composition of example 3 was applied to the skin, and a pleasant feel was able to be observed.

1. A photoprotective cosmetic composition in the form of an emulsion comprising:
a) cerium oxide secondary particles with a mean diameter d₅₀ measured with a centrifugation particle size analyzer of between 35 and 300 nm, functionalized with polyacrylic acid (PAA):
the weight-average molecular mass M_{w} of which is between 1500 and 4000 g/mol, or
comprising from 15 to 60 polymerized acrylic acid units,
said secondary particles being formed from aggregated primary particles with a mean diameter d_{TEM} of between 25 and 110 nm and a standard deviation S_{TEM} which is less than 30% of said mean diameter, d_{TEM} and S_{TEM} being calculated from a diameter distribution determined by means of transmission electron microscopy (TEM);
b) at least one inorganic UV-screening agent based on titanium oxide or zinc oxide; and
c) optionally at least one organic UV-screening agent.

2. A photoprotective cosmetic composition in the form of an emulsion comprising:
a) cerium oxide particles functionalized with polyacrylic acid (PAA);
b) at least one inorganic UV-screening agent based on titanium oxide or zinc oxide;
c) optionally at least one organic UV-screening agent, obtained from an aqueous-phase dispersion of cerium oxide secondary particles with a mean diameter d₅₀ measured with a centrifugation particle size analyzer of between and 300 nm, functionalized with polyacrylic acid (PAA):
the weight-average molecular mass M_{w} of which is between 1500 and 4000 g/mol, or
comprising from 15 to 60 polymerized acrylic acid units,
said secondary particles being formed from aggregated primary particles with a mean diameter d_{TEM} of between 25 and 110 nm and a standard deviation S_{TEM} which is less than 30% of said mean diameter, d_{TEM} and S_{TEM} being calculated from a diameter distribution determined by means of transmission electron microscopy (TEM).

3. The composition as claimed in claim 1, comprising, in a physiologically acceptable support:
i) at least one aqueous phase;
ii) at least one oily phase;
iii) at least one emulsifying system;
iv) the functionalized cerium oxide;
v) at least one inorganic UV-screening agent based on titanium oxide or zinc oxide;
vi) optionally at least one organic UV-screening agent.

4. The composition as claimed in claim 1, characterized in that the emulsion is of the oil-in-water, water-in-oil, water-in-oil-in-water or oil-in-water-in-oil type.

5. The composition as claimed in claim 1, characterized in that the emulsion is stable when the composition is subjected to each of the following tests:
4 weeks, 4° C.
4 weeks, room temperature
4 weeks, 40° C.
10 days, 50° C.
1 week, cycles: 12 hours at +20° C., then 12 hours at −10° C.
1 week, cycles: 12 hours at +40° C., then 12 hours at −10° C.

6. The composition as claimed in claim 1, characterized in that the diameter distribution of the secondary particles has a dispersion index (σ/m) of less than 0.50, preferably less than 0.35.

7. The composition as claimed in claim 1, characterized in that the secondary particles have a mean diameter d₅₀ of between 35 and 100 nm and the primary particles have a diameter d_{TEM} of about 30 nm or between 25 and 40 nm or alternatively the secondary particles have a mean diameter d₅₀ of between 75 and 200 nm and the primary particles have a diameter d_{TEM} of about 60 nm or between 60 and 120 nm.

8. The composition as claimed in claim 1, characterized in that the functionalized cerium oxide particles have a polygonal shape.

9. The composition as claimed in claim 1, characterized in that the diameter distribution d_{TEM} of the primary particles is a monopopulation.

10. The composition as claimed in claim 1, characterized in that the PAA is in acidic or basic form or partly in acidic and basic form.

11. The composition as claimed in claim 1, characterized in that the standard deviation S_{TEM} is less than 30% of the mean diameter d_{TEM}.

12. A method for preparing a photoprotective cosmetic composition according to claim 1, the method comprising mixing the functionalized cerium oxide secondary particles, or an aqueous-phase dispersion thereof, with the inorganic UV-screening agent and, optionally, the organic UV-screening agent.

13. A method for preparing a photoprotective cosmetic composition according to claim 2, the method comprising mixing the functionalized cerium oxide secondary particles, or an aqueous-phase dispersion thereof, with the inorganic UV-screening agent and, optionally, the organic UV-screening agent.

14. A process for preparing a photoprotective cosmetic composition as defined in claim 1, in which the mixture formed from an aqueous phase comprising the functionalized cerium oxide and from an oily phase comprising the inorganic UV-screening agent other than the functionalized cerium oxide is subjected to shear.

15. The composition as claimed in claim 2, comprising, in a physiologically acceptable support:
i) at least one aqueous phase;
ii) at least one oily phase;
iii) at least one emulsifying system;
iv) the functionalized cerium oxide;
v) at least one inorganic UV-screening agent based on titanium oxide or zinc oxide;
vi) optionally at least one organic UV-screening agent.

16. The composition as claimed in claim 2, characterized in that the emulsion is stable when the composition is subjected to each of the following tests:
4 weeks, 4° C.
4 weeks, room temperature
4 weeks, 40° C.
10 days, 50° C.
1 week, cycles: 12 hours at +20° C., then 12 hours at −10° C.
1 week, cycles: 12 hours at +40° C., then 12 hours at −10° C.

17. The composition as claimed in claim 2, characterized in that the diameter distribution of the secondary particles has a dispersion index (σ/m) of less than 0.50.

18. The composition as claimed in claim 2, characterized in that the secondary particles have a mean diameter d₅₀ of between 35 and 100 nm and the primary particles have a diameter d_{TEM} of about 30 nm or between 25 and 40 nm or alternatively the secondary particles have a mean diameter d₅₀ of between 75 and 200 nm and the primary particles have a diameter d_{TEM} of about 60 nm or between 60 and 120 nm.

19. The composition as claimed in claim 2, characterized in that the diameter distribution d_{TEM} of the primary particles is a monopopulation.

20. The composition as claimed in claim 2, characterized in that the standard deviation S_{TEM} is less than 30% of the mean diameter d_{TEM}.
